(19) United States
(12) Patent Application Publication
(10) Pub. No.: US 2026/0007837 A1
(43) Pub. Date: Jan. 8, 2026

(54) NEEDLE FOR ACCURATELY ADMINISTRATING ANESTHESIA IN DENTISTRY

(71) Applicant: Krystyna Blanchard, Peabody, MA (US)

(72) Inventor: Krystyna Blanchard, Peabody, MA (US)

(21) Appl. No.: 18/761,548

(22) Filed: Jul. 2, 2024

(51) Int. Cl. A61M 5/34; A61M 5/24; A61M 19/00

(52) U.S. Cl. CPC A61M 5/34; A61M 5/2425; A61M 19/00; A61M 2202/0007

(57) ABSTRACT

The present disclosure teaches a needle for accurately administrating anesthesia in dentistry. A body of the needle may include marks indicating the recommended depth/range for specific types of anesthesia. Wherein, a range may either be indicated by two marks or the same color, or by a widened band of coloring. Wherein, bright colors may be selected for the marks to maximize visual contrast against the body of the needle. Wherein, the body of the needle may also include a mark indicating a “danger zone” toward the hub end of the needle, preferably in red. Wherein, the body of the needle may also include one or more marks indicating a midpoint of the needle. Wherein, types of anesthesia belonging to the same family may be indicated by same-colored marks. Wherein, different colors may also be used to indicate a lower, mid, or higher range of the needle.

### TECHNICAL FIELD

The present disclosure relates to the field of dental appliances, or more specifically, a sterile, one-time use, marked needle for administrating anesthesia used in dentistry.

### BACKGROUND

Administrating anesthesia is a common procedure in dentistry. It is important that anesthesia is injected at an appropriate depth/range. If the needle does not go deep enough, the anesthesia may not work. If the needle goes too deep, it may cause damage to the patient's nerves.

However, when administrating anesthesia with a needle, it can be hard for the operator-especially students and less experienced professionals—to advance the needle and keep the needle at an appropriate depth. The operator may advance the needle too far, not far enough, or retract the needle while performing the injection. Some operators may even struggle to see if the bevel of the needle has penetrated an initial layer of the tissue, causing some anesthetic to drip into the patient or a student partner's mouth. This may cause unnecessary discomfort or even injury to the patient or student partner; or even worse, in some cases, the needle might break if it advances too far. Hence, it would be helpful to introduce a needle for administrating anesthesia with visual indicators on the body of the needle, which could provide visual aids to the operator to facilitate the accurate administration of anesthesia.

### SUMMARY

The presently disclosed technology teaches a needle for accurately administrating anesthesia in dentistry, comprising: a body of the needle, including a bevel on one end of the body to penetrate oral tissue and a lumen in the middle of the body for the liquid to go through; a first group of one or more marks on the body; wherein, a distance from each mark in the first group of one or more marks to the bevel indicates a recommended depth of injection for a first type of anesthesia the mark corresponds to; a second group of one or more marks on the body; wherein, the second group of one or more mark are placed at a midpoint of the body of the needle, indicating the midpoint of the body of the needle; a third group of one mark on the body; wherein, the third group of one mark is placed close to an end of the body of the needle opposite to the bevel; wherein, the third group of one mark is in red and indicates a risk of breakage of the body of the needle, a fourth group of one or more pairs of marks on the body; wherein, each pair of marks in the fourth group of one or more pairs of marks is in a same color and respectively indicates a lower boundary and an upper boundary of a recommended range of injection for a second type of anesthesia the pair of marks corresponds to; wherein, different pairs of marks in the fourth group are in different colors

In some embodiments, the needle is 32 mm long.

In some embodiments, the first group includes a mark placed at a 3 mm distance to the bevel, indicating the recommended depth of injection for Long Buccal nerve block.

In some embodiments, the second group includes one mark placed at a 15 mm distance to the bevel.

In some embodiments, the third group includes a red mark placed at a 30 mm distance to the bevel.

In some embodiments, the fourth group includes: a pair of marks of a first color respectively placed at a 14 mm distance to the bevel, and a 16 mm distance to the bevel, indicating a recommended range of injection for IO nerve block; a pair of marks of a second color respectively placed at a 20 mm distance to the bevel, and a 25 mm distance to the bevel, indicating a recommended range of injection for IA/LINGUAL nerve block.

In some embodiments, the needle is 20 mm long.

In some embodiments, the first group includes: five marks respectively placed at 3 mm, 4 mm. 5 mm, 6 mm, and 8 mm distances to the bevel, respectively indicating recommended depths of injection for Supraperiosteal, ASA, MSA, GP, and NP nerve blocks.

In some embodiments, the second group includes a mark placed at a 10 mm distance to the bevel.

In some embodiments, the third group includes a red mark placed at an 18 mm distance to the bevel.

In some embodiments, the fourth group includes a pair of marks placed at a 14 mm distance to the bevel, and a 16 mm distance to the bevel, respectively, to indicate a recommended range of injection for PSA nerve block.

In some embodiments, the presently disclosed needle further includes a visual marking on the body of the needle that indicates an orientation of the bevel.

In some embodiments, the first group of one or more marks, the second group of one or more marks, and the fourth group of one or more pairs of marks all have colors other than red.

In some embodiments, the needle is detachably connected to the head.

In some embodiments, the head is detachably connected to the chamber.

In some embodiments, the presently disclosed technology further includes a method for accurately administrating anesthesia in dentistry, comprising: identifying a target type of anesthesia; selecting a needle, wherein the needle includes: a body of the needle, including a bevel on one end of the body and a lumen in the middle of the body; a first group of one or more marks on the body; wherein, a distance from each mark in the first group of one or more marks to the bevel indicates a recommended depth of injection for a first type of anesthesia the mark corresponds to; a second group of one or more marks on the body; wherein, the second group of one or more mark are placed at or close to a midpoint of the body of the needle, indicating the midpoint of the body of the needle; a third group of one mark on the body; wherein, the third group of one mark is placed close to an end of the body of the needle opposite to the bevel; wherein, the third group of one mark is in red and indicates a risk of breakage of the body of the needle, a fourth group of one or more pairs of marks on the body; wherein, each pair of marks in the fourth group of one or more pairs of marks is in a same color and respectively indicates a lower boundary and an upper boundary of a recommended range of injection for a second type of anesthesia the pair of marks corresponds to; wherein, different pairs of marks in the fourth group are in different colors; attaching the needle to a hand-held syringe; drawing the target type of anesthesia into a chamber of the syringe by pulling a plunger of the syringe; piercing an oral mucosa with the bevel; in response to that the target type of anesthesia corresponds to a recommended depth: advancing the needle; in response to that a mark corresponding to the recommended depth levels with the oral mucosa: making the injection by pushing the plunger; in response to that the target type of anesthesia corresponds to a recommended range: advancing the needle; in response to that a first mark corresponding to a lower boundary of the recommended range is below the oral mucosa and a second mark corresponding to an upper boundary of the recommended range is above the oral mucosa: making the injection by pushing the plunger; withdrawing the needle from oral tissue.

In some embodiments, the presently disclosed technology further teaches a hand-held syringe with a needle for accurately administrating anesthesia in dentistry, comprising: a chamber to hold and store liquid; a plunger to draw the liquid into the chamber, and release the liquid from the chamber a head connecting the chamber and the needle; a body of the needle, including a bevel on one end of the body to penetrate oral tissue and a lumen in the middle of the body for the liquid to go through; a first group of one or more marks on the body; wherein, a distance from each mark in the first group of one or more marks to the bevel indicates a recommended depth of injection for a first type of anesthesia the mark corresponds to; a second group of one or more marks on the body; wherein, the second group of one or more mark are placed at or close to a midpoint of the body of the needle, indicating the midpoint of the body of the needle; a third group of one mark on the body; wherein, the third group of one mark is placed close to an end of the body of the needle opposite to the bevel; wherein, the third group of one mark is in red and indicates a risk of breakage of the body of the needle, a fourth group of one or more pairs of marks on the body; wherein, each pair of marks in the fourth group of one or more pairs of marks is in a same color and respectively indicates a lower boundary and an upper boundary of a recommended range of injection for a second type of anesthesia the pair of marks corresponds to; wherein, different pairs of marks in the fourth group are in different colors.

In some embodiments, the chamber contains a quantity of a target type of anesthesia.

### BRIEF DESCRIPTION OF THE DRAWINGS

The present disclosure is further illustrated by way of exemplary embodiments, which are described in detail through the accompanying drawings. These embodiments are not limiting, and in these embodiments, the same numbering indicates the same structure, wherein:

FIGS. 1A and 1B are diagrams illustrating the structure of two needles for accurately administrating anesthesia in dentistry, of different lengths, according to some embodiments of the presently disclosed technology.

FIG. 2 is a diagram illustrating the structure of a hand-held syringe with a needle for accurately administrating anesthesia in dentistry, according to some embodiments of the presently disclosed technology.

FIG. 3 is a diagram showing the process of administrating anesthesia to a patient with the presently disclosed needle, according to some embodiments of the presently disclosed technology.

### DETAILED DESCRIPTION

In order to more clearly illustrate the technical solutions of the embodiments of the present disclosure, the accompanying drawings for the description of the embodiments are described below. Obviously, the accompanying drawings in the following description are only some examples or embodiments of the present disclosure, and it is possible for a person of ordinary skill in the art to apply the present disclosure to other similar scenarios in accordance with these accompanying drawings without creative labor. Unless obviously obtained from the context or the context illustrates otherwise, the same numeral in the drawings refers to the same structure or operation.

It should be understood that the terms “system,” “device,” “unit,” and/or “module” are used herein as a way to distinguish between different components, elements, parts, sections, or assemblies at different levels. However, if other words may achieve the same purpose, the terms may be replaced with alternative expressions.

As indicated in the present disclosure and in the claims, unless the context clearly suggests an exception, the words “one,” “a,” “a kind of,” and/or “the” do not refer specifically to the singular but may also include the plural. In general, the terms “include” and “comprise” suggest only the inclusion of clearly identified steps and elements, which do not constitute an exclusive list, and the method or device may also include other steps or elements.

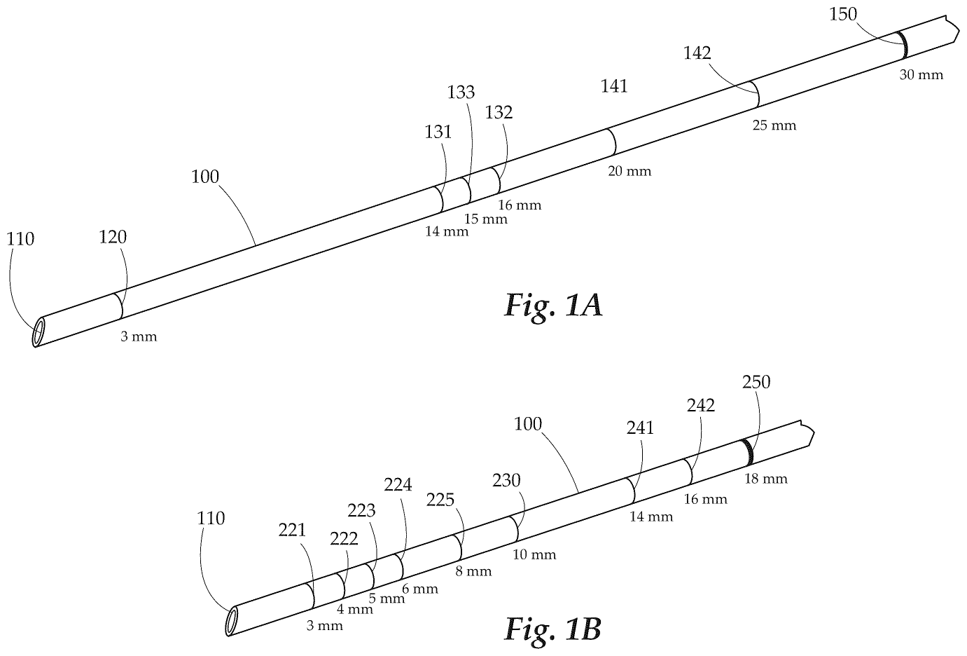

FIGS. 1A & 1B are diagrams illustrating the structure of two needles for accurately administrating anesthesia in dentistry, of different lengths, according to some embodiments of the presently disclosed technology.

In one preferred embodiment, two needles of different lengths are respectively used for injections of different types of anesthesia requiring different preferred depths. Wherein, in one preferred embodiment, the long needle may be 32 mm long, and the short needle may be 20 mm long. In some other embodiments, the length of the needle may be different. For example, the length of the needle may be shorter for treatment of a child. For another example, the length of the needle may be different for injections at different locations on the upper jaw and the lower jaw.

In some embodiments, the body of the needle may include a number of marks to indicate the preferred depths of injection for different types of anesthesia, as well as giving warnings and visual aids. In some embodiments, different types of marks may have different colors. In some embodiments, bright colors, such as yellow, blue, or red, may be used for the marks, to maximize visual contrast against the body of the needle, which may usually be silver and reflective, thus offering greater visibility. In some embodiments, the marks may be implemented as a layer of colored materials over the needle.

The choice of colors may serve different indicative purposes. For example, marks indicating recommended depths of injection for types of anesthesia belonging to the same family may be in the same color, and marks for types of anesthesia belonging to different families may be in different colors. For another example, marks indicating recommended depths of injection, marks indicating a midpoint of the needle, and marks indicating a “danger zone” of the needle (the needle has been advanced too far and it thus risks breakage) may be in different colors. For yet another example, marks indicating the “danger zone” may be in red, since red is a bright and alerting color to human eyes and is usually associated with danger and forbiddingness. In some embodiments, the entire end range, such as the last few millimeters on the body of the needle, may be colored in red.

Besides a recommended depth for injection, marks may also be used to indicate a recommended range for injection. For example, two marks of the same color may be respectively placed at the lower boundary and the higher boundary to indicate the preferred range corresponding to a type of anesthesia. For another example, a widened band of color may be included on the body of the needle to indicate the recommended range.

For yet another example, marks of different colors may be used to indicate different parts of the needle. In some embodiments, for example, marks on a lower part of the needle may be in a first color, and marks on an upper part of the needle may be in a second color. Therefore, an operator could easily see how much the needle has advanced from the colors.

FIG. 1A illustrates the structure of the long needle, according to some embodiments of the preferred technology. As discussed above, in one preferred embodiment, the body 100 of the long needle may be 32 mm long. The body 100 may include a bevel 110 at one end to penetrate oral mucosa and tissues. The body 100 may include a lumen for liquid to go through.

As shown in FIG. 1A, in one preferred embodiment, a blue mark 120 may be included at 3 mm along the length of the body 100 of the needle to indicate a recommended depth for Long Buccal nerve block injection. Wherein, the Long Buccal nerve block may be administrated as a continuation of the IA (Inferior Alveolar)/LINGUAL nerve block. Hence, the entire process of administrating IA/LINGUAL nerve block and the Long Buccal nerve block may be completed with one needle, thus minimizing the need for needle changes and unnecessary needle movements, which might cause unwanted needle sticks or cross-contamination. Moreover, yellow marks 131 and 132 may be placed at 14 mm and 16 mm respectively to indicate recommended depths for IO nerve block injections. The yellow marks 131 and 132 may also help an operator visualize the halfway point of the needle. Alternatively, in some embodiments, a mark may be added at 15 mm to indicate the halfway point of the needle. Also, green marks 141 and 142 may be placed at 20 mm and 25 mm to indicate recommended depths for IA/LINGUAL nerve block injections. Finally, a red mark 150 may be placed at 30 mm to indicate the danger zone of the needle near the hub, which presents the highest risk for needle breakage if approached.

FIG. 1B illustrates the structure of the short needle, according to some embodiments of the preferred technology. As discussed above, in one preferred embodiment, the body 100 of the long needle may be 20 mm long. The body 100 may also include a bevel 110 at one end to penetrate oral mucosa and tissues. The body 100 may also include a lumen for liquid to go through.

As shown in FIG. 1B, in one preferred embodiment, blue marks 221, 222, 223, 224, and 225 may be respectively placed at 3 mm, 4 mm. 5 mm, 6 mm, and 8 mm from the bevel along the body 200 of the short needle to indicate recommended injection depths for the following types of anesthesia: Supraperiosteal, ASA (Anterior Superior Alveolar), MSA (Middle Superior Alveolar), GP (Greater Palatine), and NP (Nasopalatine) nerve blocks. A green mark 230 may be placed at 10 mm along the body 200 of the short needle to help visualize the midpoint of the short needle. Yellow marks 241 and 242 may be placed at 14 mm and 16 mm respectively to indicate the recommended range for PSA (Posterior Superior Alveolar) nerve block injection. As discussed above, the marks 241 and 242 may respectively indicate the higher and lower boundaries of the recommended range. Last, a red mark 250 may be placed at 18 mm to indicate the “danger zone” near the hub, and the needle may face a risk of breakage and injuring a patient if the operator keeps advancing.

Alternatively, in some embodiments, the marks may be placed at different lengths, and the coloring of the marks may be different. For example, other types of anesthesia may be adopted, and they may have different recommended depths of injection. For another example, a shorter needle may be used for babies and young kids, and the preferred depths of anesthesia injection may also be smaller. For yet another example, in some embodiments, a widened band of color may be used to indicate a preferred range of injection, instead of two narrow markings of the same color indicating the higher and lower boundaries of the range.

In some embodiments, the presently disclosed needle may also have a clear coat placed over the exterior of the needle to create a smoothness over the markings. This would ensure a smooth gliding through the soft tissues of the mouth and minimize any snagging on the markings.

In some embodiments, the needle may be sterile, one-time use, and disposable needles.

In some embodiments, the presently disclosed needle may further include a bevel indicator, which may be a visual marking on the body of the needle that indicates the orientation of the bevel. In some embodiments, the colored marks themselves may function together as a bevel indicator, as the coloring hints at the orientation of the needle. For example, the end with a red mark indicates the hub end, which is the opposite of the bevel end. In some embodiments, the bevel indicator may also be a visual marking on a package of the needle.

In some embodiments, the presently disclosed needle may be manufactured sterile and packaged in a sterile container. In some embodiments, the presently disclosed needle may be disposable.

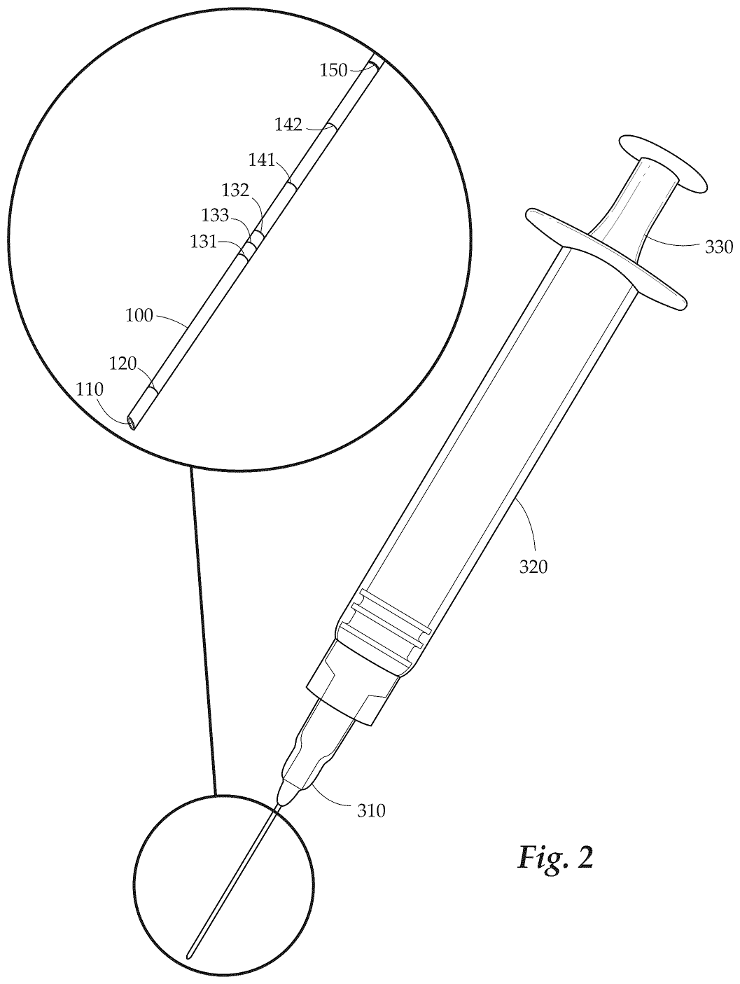

FIG. 2 is a diagram illustrating the structure of a hand-held syringe with a needle for accurately administrating anesthesia in dentistry, according to some embodiments of the presently disclosed technology.

As shown in FIG. 2, the hand-held syringe includes a head 310, a chamber 320, and a plunger 330. The head 310 may be connected to a needle 100, which was discussed in the above sections. The connection between the head 310 and the needle 100 may be detachable, or the needle 100 may be permanently connected to the head 310. The chamber 320 may be used to store liquid. In some embodiments, if the operator pulls the plunger 330, liquid may be drawn into the chamber 320; if the operator pushes the plunger, liquids may be released from the chamber through the lumen in the needle 100.

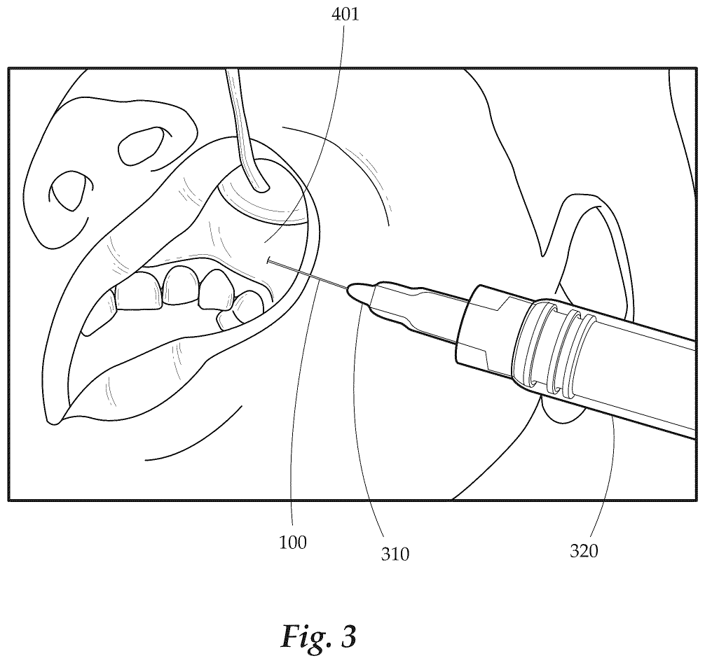

FIG. 3 is a diagram showing the process of administrating anesthesia to a patient with the presently disclosed needle, according to some embodiments of the presently disclosed technology.

In some embodiments, an operator may first attach a needle 100 to the head 310 of the hand-held syringe. The operator may select the needle based on the specific needs of the injection; for example, if the injection is on the upper jaw or the lower jaw, which anesthesia shall be used, the patient's age, etc. They may then draw anesthesia into the chamber 320 by pulling the plunger 330. After the operator penetrates the oral mucosa 401 with the bevel of the needle 100, they may pay close attention to the marks on the body of the needle while slowly advancing the needle. When the mark indicating the recommended depth for the specific type of anesthesia they are using levels with the oral mucosa 401, they may make the injection by pushing the plunger 330 and releasing the liquid from the chamber 320. Or, if a recommended range for the type of anesthesia is given by a couple of markings of the same color, the operator may make the injection when they are able to see the higher range marking above the oral mucosa 401, but not the lower range. Or, if a widened color band is used to indicate a recommended range corresponding to the type of anesthesia, the operator may make the injection when a part of the widened color band levels with the oral mucosa 401. An operator may also pay special attention to the red mark indicating the “danger zone” of injection. If the needle reaches a point where the operator is no longer able to see the red mark above the mucosa 401, the operator may be warned and stop advancing the needle.

Moreover, operators may be able to document precise depths for each patient with visual aids provided by the marks, so that they may learn each patient's skull anatomy better.

In summary, the presently disclosed technology enables operators to administrate anesthesia to a dental patient with precision and case, thus minimizing discomfort or trauma to the tissues from over-insertion of the needle and maximizing the safety of operation. The presently disclosed technology may also serve teaching purposes to dental students.

Furthermore, unless explicitly stated in the claims, the use of order, numbers, letters, or other names for processing elements and sequences are not intended to limit the order of the processes and methods of the present disclosure. While various examples have been discussed in the disclosure as currently considered useful embodiments of the invention, it should be understood that such details are provided for illustrative purposes only. The appended claims are not limited to the disclosed embodiments, and instead, the claims are intended to cover all modifications and equivalent combinations within the scope and essence of the embodiments disclosed in the present disclosure. For example, although the described system components may be implemented through a hardware device, they may also be realized solely through a software solution, such as installing the described system on an existing processing or mobile device.

Similarly, it should be noted that, for the sake of simplifying the presentation of embodiments disclosed in the present disclosure and aiding in understanding one or more embodiments of the present disclosure, various features have been sometimes combined into a single embodiment, drawing, or description. However, this manner of disclosure does not imply that the features required by the claims are more than the features mentioned in the claims. In fact, the features of the embodiments are less than all the features of the single embodiment disclosed in the foregoing disclosure.

In some embodiments, numeric values describing the composition and quantity of attributes are used in the description. It should be understood that such numeric values used for describing embodiments may be modified with qualifying terms such as “about,” “approximately” or “generally”. Unless otherwise stated, “about,” “approximately” or “generally” indicates that a variation of ±20% is permitted in the described numbers. Accordingly, in some embodiments, the numerical parameters used in the disclosure and claims are approximations, which can change depending on the desired characteristics of the individual embodiment. In some embodiments, the numerical parameters should take into account a specified number of valid digits and employ a general manner of bit retention. Although the numerical ranges and parameters used in some embodiments of the present disclosure to confirm the breadth of the range are approximations, in specific embodiments, such numerical values are set as precisely as practicable.

With respect to each of the patents, patent applications, publications of patent applications, and other material, such as articles, books, specifications, publications, documents and the like, cited in the present disclosure, the entire contents thereof are hereby incorporated herein by reference. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and the contents described herein, the descriptions, definitions, and/or use of terminology in the present disclosure shall prevail.

In closing, it should be understood that the embodiments described in the present disclosure are used only to illustrate the principles of the embodiments of the present disclosure. Other deformations may also fall within the scope of the present disclosure. Therefore, by way of example and not limitation, alternative configurations of the embodiments disclosed in the present disclosure may be considered consistent with the teachings of the present disclosure. Accordingly, the embodiments described in the present disclosure are not limited to the explicitly introduced and described embodiments in the present disclosure.

What is claimed is:

1. A needle for accurately administrating anesthesia in dentistry, comprising:
a body of the needle, including a bevel on one end of the body to penetrate oral tissue and
a lumen in the middle of the body for the liquid to go through;
a first group of one or more marks on the body;
wherein, a distance from each mark in the first group of one or more marks to the bevel indicates a recommended depth of injection for a first type of anesthesia the mark corresponds to;
a second group of one or more marks on the body;
wherein, the second group of one or more mark are placed at a midpoint of the body of the needle, indicating the midpoint of the body of the needle;
a third group of one mark on the body;
wherein, the third group of one mark is placed close to an end of the body of the needle opposite to the bevel;
wherein, the third group of one mark is in red and indicates a risk of breakage of the body of the needle,
a fourth group of one or more pairs of marks on the body;
wherein, each pair of marks in the fourth group of one or more pairs of marks is in a same color and respectively indicates a lower boundary and an upper boundary of a recommended range of injection for a second type of anesthesia the pair of marks corresponds to;
wherein, different pairs of marks in the fourth group are in different colors.

2. The needle as in claim 1, wherein the needle is 32 mm long.

3. The needle as in claim 2, wherein the first group includes a mark placed at a 3 mm distance to the bevel, indicating the recommended depth of injection for Long Buccal nerve block.

4. The needle as in claim 2, wherein the second group includes one mark placed at a 15 mm distance to the bevel.

5. The needle as in claim 2, wherein the third group includes a red mark placed at a 30 mm distance to the bevel.

6. The needle as in claim 2, wherein the fourth group includes:
a pair of marks of a first color respectively placed at a 14 mm distance to the bevel, and a 16 mm distance to the bevel, indicating a recommended range of injection for IO nerve block;
a pair of marks of a second color respectively placed at a 20 mm distance to the bevel, and a 25 mm distance to the bevel, indicating a recommended range of injection for IA/LINGUAL nerve block.

7. The needle as in claim 2, wherein the needle is 20 mm long.

8. The needle as in claim 7, wherein the first group includes:
five marks respectively placed at 3 mm, 4 mm. 5 mm, 6 mm, and 8 mm distances to the bevel, respectively indicating recommended depths of injection for Supraperiosteal, ASA, MSA, GP, and NP nerve blocks.

9. The needle as in claim 7, wherein the second group includes a mark placed at a 10 mm distance to the bevel.

10. The needle as in claim 7, wherein the third group includes a red mark placed at an 18 mm distance to the bevel.

11. The needle as in claim 7, wherein the fourth group includes a pair of marks placed at a 14 mm distance to the bevel, and a 16 mm distance to the bevel, respectively, to indicate a recommended range of injection for PSA nerve block.

12. The needle as in claim 1, further comprising:
a visual marking on the body of the needle that indicates an orientation of the bevel.

13. The needle as in claim 1, wherein the first group of one or more marks, the second group of one or more marks, and the fourth group of one or more pairs of marks all have colors other than red.

14. The needle as in claim 1, wherein the needle is detachably connected to the head.

15. The needle as in claim 1, wherein the head is detachably connected to the chamber.

16. A method for accurately administrating anesthesia in dentistry, comprising:
identifying a target type of anesthesia;
selecting a needle, wherein the needle includes:
a body of the needle, including a bevel on one end of the body and a lumen in the middle of the body;
a first group of one or more marks on the body;
wherein, a distance from each mark in the first group of one or more marks to the bevel indicates a recommended depth of injection for a first type of anesthesia the mark corresponds to;
a second group of one or more marks on the body;
wherein, the second group of one or more mark are placed at or close to a midpoint of the body of the needle, indicating the midpoint of the body of the needle;
a third group of one mark on the body;
wherein, the third group of one mark is placed close to an end of the body of the needle opposite to the bevel;
wherein, the third group of one mark is in red and indicates a risk of breakage of the body of the needle,
a fourth group of one or more pairs of marks on the body;
wherein, each pair of marks in the fourth group of one or more pairs of marks is in a same color and respectively indicates a lower boundary and an upper boundary of a recommended range of injection for a second type of anesthesia the pair of marks corresponds to;
wherein, different pairs of marks in the fourth group are in different colors;
attaching the needle to a hand-held syringe;
drawing the target type of anesthesia into a chamber of the syringe by pulling a plunger of the syringe;
piercing an oral mucosa with the bevel;
in response to that the target type of anesthesia corresponds to a recommended depth:
advancing the needle;
in response to that a mark corresponding to the recommended depth levels with the oral mucosa:
making the injection by pushing the plunger;
in response to that the target type of anesthesia corresponds to a recommended range:
advancing the needle;
in response to that a first mark corresponding to a lower boundary of the recommended range is below the oral mucosa and a second mark corresponding to an upper boundary of the recommended range is above the oral mucosa:
making the injection by pushing the plunger;
withdrawing the needle from oral tissue.

17. A hand-held syringe with a needle for accurately administrating anesthesia in dentistry, comprising:
a chamber to hold and store liquid;
a plunger to draw the liquid into the chamber, and release the liquid from the chamber;
a head connecting the chamber and the needle;
a body of the needle, including a bevel on one end of the body to penetrate oral tissue and
a lumen in the middle of the body for the liquid to go through;
a first group of one or more marks on the body;
wherein, a distance from each mark in the first group of one or more marks to the bevel indicates a recommended depth of injection for a first type of anesthesia the mark corresponds to;
a second group of one or more marks on the body;
wherein, the second group of one or more mark are placed at or close to a midpoint of the body of the needle, indicating the midpoint of the body of the needle;
a third group of one mark on the body;
wherein, the third group of one mark is placed close to an end of the body of the needle opposite to the bevel;
wherein, the third group of one mark is in red and indicates a risk of breakage of the body of the needle,
a fourth group of one or more pairs of marks on the body;
wherein, each pair of marks in the fourth group of one or more pairs of marks is in a same color and respectively indicates a lower boundary and an upper boundary of a recommended range of injection for a second type of anesthesia the pair of marks corresponds to;
wherein, different pairs of marks in the fourth group are in different colors.

18. The hand-held syringe as in claim 20, wherein the chamber contains a quantity of a target type of anesthesia.
